(19) United States
(12) Patent Application Publication
(10) Pub. No.: US 2026/0130917 A1
(43) Pub. Date: May 14, 2026

(54) ANTIVIRAL COMPOSITION FOR CORONAVIRUS OR INFLUENZA VIRUS, USING NATURAL PRODUCTS, AND USE THEREOF

(71) Applicants: KOREA ADVANCED INSTITUTE OF SCIENCE AND TECHNOLOGY, Daejeon (KR); INSTITUT PASTEUR KOREA, Gyeonggi-do (KR)

(72) Inventors: Sang Yup LEE, Daejeon (KR); Woo Dae JANG, Daejeon (KR); Seungtaek KIM, Gyeonggi-do (KR); Sangeun JEON, Gyeonggi-do (KR); Hyo Jung LEE, Gyeonggi-do (KR)

(21) Appl. No.: 19/118,628

(22) PCT Filed: Oct. 5, 2023
(86) PCT No.: PCT/KR2023/015331

§ 371 Date: Apr. 4, 2025

(30) Foreign Application Priority Data
Oct. 5, 2022 (KR) 10-2022-0127097

(51) Int. Cl. A61K 31/519; A61P 31/16

(52) U.S. Cl. CPC A61K 31/519; A61P 31/16

(57) ABSTRACT

The present invention relates to an antiviral composition for coronavirus or influenza virus, using a natural product, and use thereof. The antiviral composition for coronavirus or influenza virus and a pharmaceutical composition for preventing or treating the viral infection thereof, according to the present invention, are based on a natural product or a natural product derivative so as to be less toxic than a conventional synthetic drug, and have already been consumed as food, thereby verifying empirical safety and effectiveness, and thus are useful as a safer and more effective antiviral and viral infection prevention or treatment.

### TECHNICAL FIELD

The present invention relates to an anti-viral composition against coronavirus or influenza virus using natural products, and more specifically, to an anti-viral composition against coronavirus or influenza virus using natural products or natural product derivatives using virtual drug screening technology, and a pharmaceutical composition for preventing or treating coronavirus infection or influenza.

### BACKGROUND ART

Coronaviruses are enveloped, single-stranded positive-sense RNA viruses with a genome size of 25 to 22 kb, and are relatively large viruses among RNA viruses known so far. Coronavirus has a specific structure having a halo- or crown-like appearance due to club-shaped protrusions that stick out of the viral envelopes, and took their name from “corona” which means crown in Latin. Coronaviruses that have been found in various birds and animals such as bats, birds, cats, dogs, cows, pigs and mice since first found in chickens in 1937 are divided into four groups (alpha-, beta-gamma-, and delta-corona viruses). The alpha- and beta-coronavirus groups primarily infect mammals, and the gamma- and delta-coronavirus groups can be found in birds. Coronaviruses are known to cause various diseases such as gastrointestinal and respiratory diseases in animals. Human coronaviruses that infect humans include HCoV-229E and HCoV-OC43 discovered in the 1960s, and HCoV-NL63 (2004) and HCoV-HKU1 (2005) discovered after the SARS pandemic. Although these coronaviruses are generally known to be related to upper respiratory tract infections, they may also cause serious lung diseases in immunodeficient patients. It has been reported that the infection rate for coronavirus increases mainly in winter or early spring, and it is known that the proportion of adult cold patients caused by coronavirus as a pathogen is quite high.

Coronavirus disease 2019 is a viral respiratory disease that occurred in Wuhan, China in December 2019. It is also called ‘Wuhan pneumonia’, ‘novel coronavirus infection’, or ‘COVID-19’. It is a pandemic disease caused by a novel coronavirus, which infects through the respiratory tract, and shows a highly contagious characteristic at the beginning of infection with few symptoms. After infection, coronavirus disease 2019 develops into pneumonia after symptoms such as sore throat, high fever, cough and difficulty breathing. As coronavirus disease 2019 began to spread worldwide, the World Health Organization declared a pandemic of this disease in March 2020. Several mutant SARS-CoV-2 viruses have emerged to date and typical mutant viruses are as follows (Ancestral SARS-CoV-2 (lineage A_China), Alpha (B.1.1.7_UK), Beta (B.1.351_South Africa), Gamma (P.1_Brazil), Delta (B.1.617.2_India), Omicron (B.1.1.529)).

Coronavirus disease 2019 (COVID-19) mainly attacks the respiratory tract. When infected, the virus invades the lungs, and symptoms such as high fever, cough and difficulty breathing occur, and symptoms similar to pneumonia appear, and in severe cases, the alveoli are damaged, leading to death due to respiratory failure. The incubation period of coronavirus disease 2019 is 3 to 7 days, but it can last up to 14 days. On Jan. 30, 2020, China announced that there were cases where the incubation period had increased to 23 days. It has been reported that coronavirus disease 2019 spreads even during the incubation period when symptoms do not appear.

Severe acute respiratory syndrome coronavirus 2 (SARS-CoV-2) is a virus that causes coronavirus disease 2019. Human-to-human transmission of the virus has been confirmed in the academic world, and this coronavirus is spread mainly through close contact with respiratory droplets from cough or runny nose, especially within a 2 m radius (https://www.cdc.gov/coronavirus/2019-ncov/about/transmission.html). Touching the eyes, nose or mouth after touching a surface or object contaminated with the virus is another cause of coronavirus disease 2019 infection. The RNA of the virus was also found in stool samples from infected patients (ML. Holshue et al., N Engl J Med 2020; 382:929-936).

The entry of SARS-CoV-2 into the cell is mediated by the binding of the spike (S) viral protein, a 1273 amino acid long protein which belongs to the viral envelope and protrudes outwards with a ‘corona’ like appearance, to angiotensin converting enzyme 2 (ACE2) receptors. ACE2 mediates cell entry of three coronavirus strains (SARS-CoV, NL63 and SARSCoV-2), and in particular, SARS-CoV and SARS-CoV2 share 76% identity in the amino acid sequence, thereby explaining the tendency of these viruses to bind to ACE2. The first step of the viral entry process is the binding of the N-terminal portion of the viral protein unit S1 to the pocket of the ACE2 receptor. The second step, which is believed to be of utmost importance for viral entry, is protein cleavage between the S1 and S2 units, which is mediated by the receptor transmembrane protease serine 2 (TMPRSS2), a member of the Hepsin/TMPRSS subfamily (Polo V. et al., European Journal of Internal Medicine, 2020).

Virtual drug screening technology aims to rapidly construct a compound library composed of active compounds, which are expected to be effective, through a computer prior to production of an actual compound library. Virtual drug screening is usually performed by sequentially applying various strategies such as ligand-based screening, pharmacophore-based screening, and protein structure-based screening. In ligand-based screening, compounds with high two-dimensional or three-dimensional structural similarity are searched for in a database using the structure of an active compound, which is already known to be active against a drug target, as a reference. Pharmacophore-based screening is a method of screening compounds having similar three-dimensional arrangement information about specific functional groups of molecules that exhibit pharmacological action. Protein structure-based screening is a method of evaluating candidates by calculating the affinity of a compound for a drug target through docking simulation. As an evaluation function for calculating energy in this process, a force-field based method, an empirical method, a knowledge-based method, a consensus scoring method, etc. may be used.

Coronavirus disease 2019 caused more than 540 million confirmed cases and more than 6.3 million deaths worldwide until June 2022, and is posing a fatal threat to human health. Currently, drugs such as remdesivir, Paxlovid, and molnupiravir have been approved by the US FDA for the treatment of COVID-19, but clinical results have fallen short of expectations due to issues such as drug interactions, efficacy, and side effects.

Remdesivir is an antiviral drug prepared from a specific nucleotide analog prodrug. It was originally developed by Gilead Sciences as a drug to treat Ebola hemorrhagic fever and Marburg virus, but has been shown to have antiviral effects on single-stranded RNA viruses such as respiratory syncytial virus, Junin virus, Lassa fever virus, henipavirus, and coronavirus (including MERS and SARS viruses) in several experiments (Michael K. Lo et al., Scientific reports, Vol. 7, pp. 43395, 2017).

Under this background, as a result of extensive efforts to solve the problems and develop a drug for treating coronavirus infection, the present inventors conceived a substance that binds to the Spike of SARS-CoV-2 and inhibits the entry of the virus into host cells, and found that the derived candidate substance exhibits an anti-SARS-CoV-2 effect when primate cells (Vero) and human lung cells, Calu-3 are treated with the candidate substance in combination with the SARS-CoV-2 virus. In addition, the present inventors found that the derived candidate substance exhibits an excellent proliferation inhibitory effect not only against SARS-CoV-2 but also against both influenza A and B viruses, and completed the present invention.

The above information disclosed in this Background section is only for enhancement of understanding of the background of the present invention. Therefore, it may not contain information that forms the conventional art that is already known in the art to which the present invention pertains.

### DISCLOSURE

Therefore, the present invention has been made in view of the above problems, and it is an object of the present invention to provide an anti-viral composition against coronavirus or influenza virus using a natural product and the use thereof.

It is another object of the present invention to provide a composition for preventing or treating coronavirus infection or influenza containing the natural product and the use thereof.

In accordance with an aspect of the present invention, the above and other objects can be accomplished by the provision of an anti-viral composition against coronavirus or influenza virus containing, as an active ingredient, a compound selected from the group consisting of the following compounds, a derivative thereof or a salt thereof:
a) [3-(2,2-dimethyloxan-4-yl)-6-methylheptyl]({[4-(propan-2-yloxy)phenyl]methyl})amine and oxalic acid;
b) 10,10-dimethyl-10a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
   f) 3-(3,5-di-tert-butyl-4-hydroxyphenyl)propyl 2-(phenylamino)acetate;
   g) N-[(4-fluorophenyl)methyl]-2-[(3R,4S)-3-{[5-(phenoxymethyl)-1,2-oxazol-3-yl]methyl}piperidin-4-yl]acetamide;
   h) 4-[(heptyloxy)carbonyl]phenyl pyridine-3-carboxylate;
   i) 2-octyl-1,3-dioxan-5-yl 4-methoxybenzoate;
   j) N,1-bis(2-phenylethyl)-9H-pyrido[3,4-b]indole-3-carboxamide;
   k) (4Z,7E,10E,13E,16E,19E)-docosa-4,7,10,13,16,19-hexaenoic acid;
   l) [(1R,9aR)-octahydro-1H-quinolizin-1-yl]methyl 5-[(benzylsulfanyl)methyl]furan-2-carboxylate;
   m) 4-oxo-8-propyl-1H,2H,3H,4H-cyclopenta[c]chromen-7-yl 2-{[(benzyloxy)carbonyl]amino}acetate;
   n) Tricosa-10,12-dienoic acid;
   o) Ethyl 2-tert-butyl-5-(4-methoxybenzoyloxy)-1-benzofuran-3-carboxylate;
   p) 3-(3,5-di-tert-butyl-4-hydroxyphenyl)propyl 2-(cyclohexylamino)acetate;
   q) (5Z,8Z,11Z,14Z,17Z)-icosa-5,8,11,14,17-pentaenoic acid;
   r) 4-(4-heptylbenzoyloxy)benzoic acid;
   s) 4-[(heptyloxy)carbonyl]phenyl 5-bromopyridine-3-carboxylate;
   t) (9Z,12Z,15Z)-octadeca-9,12,15-trienoic acid;
   u) (4Z,7Z,10Z,13Z,16Z,19Z)-docosa-4,7,10,13,16,19-hexaenoic acid;
   v) (9Z,12R)-12-hydroxyoctadec-9-enoic acid;
   w) (9Z)-octadec-9-enoic acid;
   x) 4-[(1-benzofuran-2-yl)formamido]-N-[2-(1H-indol-3-yl)ethyl]butanamide; and
   y) 4-(methylsulfanyl)phenyl 4-heptylbenzoate.

In accordance with another aspect of the present invention, there is provided anti-viral use of the compound selected from the group consisting of the compounds for a coronavirus or influenza virus.

In accordance with another aspect of the present invention, there is provided use of the compound selected from the group consisting of the compounds for preparation of an anti-viral composition against a coronavirus or influenza virus.

In accordance with another aspect of the present invention, there is provided a method of inhibiting infection, activity and/or proliferation of a coronavirus or influenza virus, including treatment with the compound selected from the group consisting of the compounds.

In accordance with another aspect of the present invention, there is provided a pharmaceutical composition for preventing or treating a coronavirus infection or influenza containing the compound selected from the group consisting of the compounds, the derivative thereof or the pharmaceutically acceptable salt thereof.

In accordance with another aspect of the present invention, there is provided use of the compound selected from the group consisting of the compounds for prevention or treatment of a coronavirus infection or influenza.

In accordance with another aspect of the present invention, there is provided use of the compound selected from the group of the compounds for preparation of a pharmaceutical composition for preventing or treating a coronavirus infection or influenza.

In accordance with another aspect of the present invention, there is provided a method of preventing or treating coronavirus infection or influenza including administering to a subject the compound selected from the group of the compounds.

### DESCRIPTION OF DRAWINGS

FIG. 1 illustrates a drug virtual screening procedure used in the present invention.

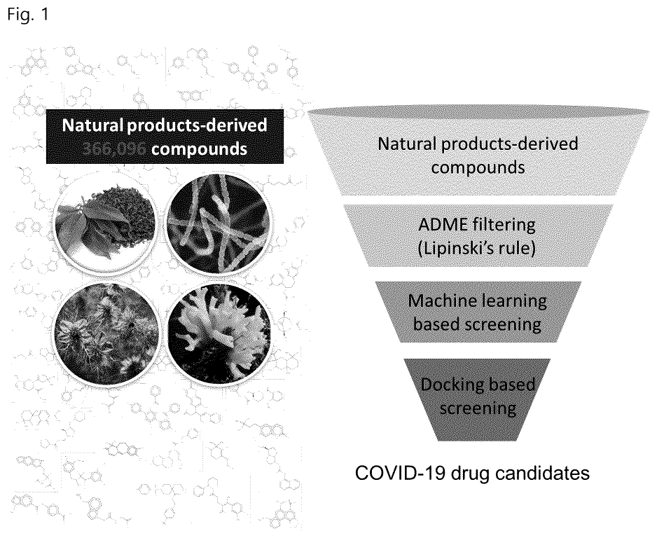

FIG. 2 illustrates the spike protein structure of SARS-CoV-2, the drug target used in the present invention.

FIG. 3 illustrates the drug binding pocket present in the receptor-binding domain of the SARS-CoV-2 spike protein structure, the drug target used in the present invention.

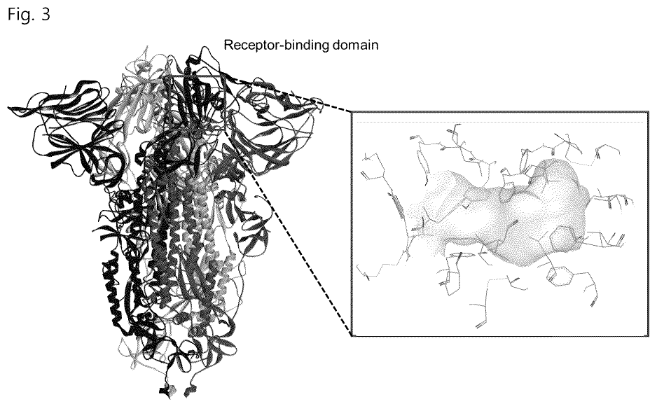

FIG. 4 shows the results of antiviral activity in monkey kidney cells for natural products or natural product-like compounds selected through the virtual drug screening procedure.

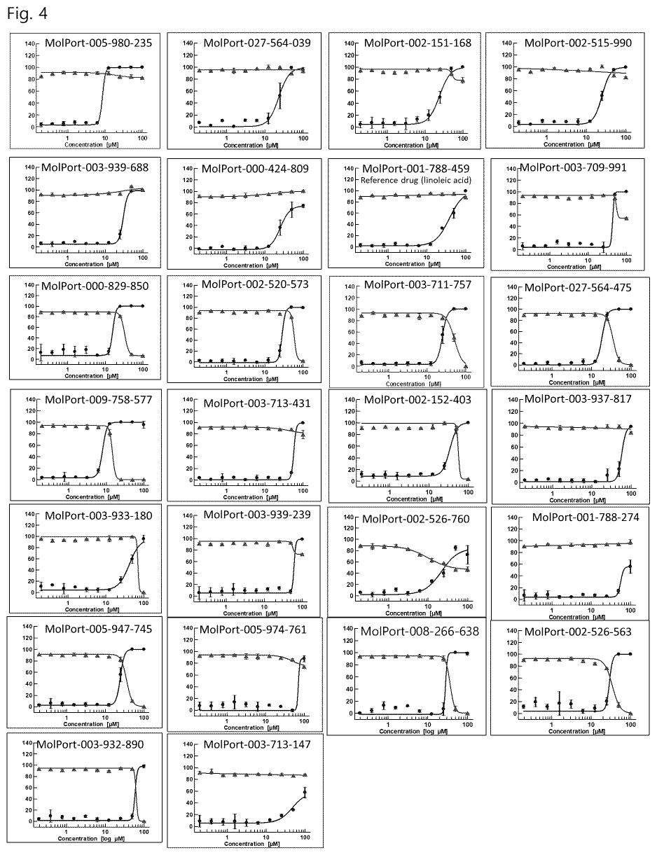

FIG. 5 shows the results of a pseudovirus assay to verify the viral entry inhibition mechanism of MolPort-005-980-235 (C-55).

FIG. 6 shows the results of a time-of-addition assay to verify the viral entry inhibition mechanism of MolPort-005-980-235 (C-55).

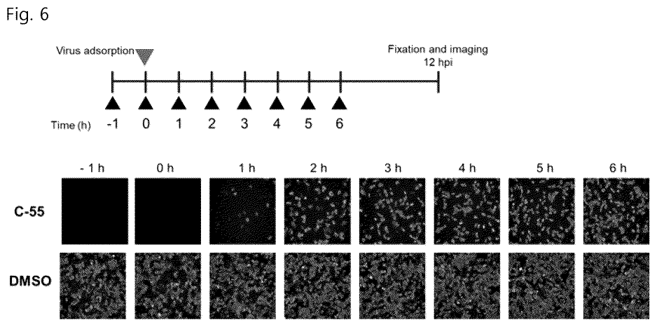

FIG. 7 shows results of reduction of viral titers and viral RNA in the lungs of SARS-CoV-2 infected mice by MolPort-005-980-235 (C-55).

FIG. 8 shows the antiviral activity of MolPort-005-980-235 (C-55) against mutant coronaviruses including Omicron in a cell-level experiment.

FIG. 9 shows the antiviral activity of MolPort-005-980-235 (C-55) against influenza A virus (H1N1 A/California/07/2009) at the cell level.

FIG. 10 shows the antiviral activity of MolPort-005-980-235 (C-55) against influenza A virus (H3N2 A/Hong Kong/4801/2014) at the cell level.

FIG. 11 shows the antiviral activity of MolPort-005-980-235 (C-55) against influenza B virus (B/Brisbane/60/2008) at the cell level.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

In one embodiment of the present invention, virtual screening was performed on binding to the spike protein of the SARS-CoV-2 virus, using ADME evaluation using Linpinski's rule, machine learning-based antiviral activity evaluation, and molecular docking simulation-based evaluation modules based on the three-dimensional structures of 366,096 natural compounds collected from MolPort, Super Natural II, MIBiG, and PubChem databases, and the final candidates were selected based on the docking binding energy between the compound candidates and the spike protein.

In another embodiment of the present invention, it was found that the selected compounds inhibit the intracellular entry and proliferation of coronaviruses and thus exhibit excellent antiviral activity, and that the selected compounds exhibit excellent antiviral activity against various mutant viruses of SARS-CoV-2.

Furthermore, in another embodiment of the present invention, it was found that the selected compounds exhibit excellent antiviral activity not only against coronaviruses but also against both influenza A and B.

Therefore, the present invention is directed to an anti-viral composition against coronavirus or influenza virus containing a compound selected from the group consisting of the following compounds, a derivative thereof or a salt thereof:
a) [3-(2,2-dimethyloxan-4-yl)-6-methylheptyl]({[4-(propan-2-yloxy)phenyl]methyl})amine and oxalic acid;
b) 10,10-dimethyl-10a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
g) N-[(4-fluorophenyl)methyl]-2-[(3R,4S)-3-{[5-(phenoxymethyl)-1,2-oxazol-3-yl]methyl}piperidin-4-yl]acetamide;
h) 4-[(heptyloxy)carbonyl]phenyl pyridine-3-carboxylate;
i) 2-octyl-1,3-dioxan-5-yl 4-methoxybenzoate;
j) N,1-bis(2-phenylethyl)-9H-pyrido[3,4-b]indole-3-carboxamide;
k) (4Z,7E,10E,13E,16E,19E)-docosa-4,7,10,13,16,19-hexaenoic acid;
l) [(1R,9aR)-octahydro-1H-quinolizin-1-yl]methyl 5-[(benzylsulfanyl)methyl]furan-2-carboxylate;
m) 4-oxo-8-propyl-1H,2H,3H,4H-cyclopenta[c]chromen-7-yl 2-{[(benzyloxy)carbonyl]amino}acetate;
n) Tricosa-10,12-dienoic acid;
o) Ethyl 2-tert-butyl-5-(4-methoxybenzoyloxy)-1-benzofuran-3-carboxylate;
p) 3-(3,5-di-tert-butyl-4-hydroxyphenyl)propyl 2-(cyclohexylamino)acetate;
q) (5Z,8Z,11Z,14Z,17Z)-icosa-5,8,11,14,17-pentaenoic acid;
r) 4-(4-heptylbenzoyloxy)benzoic acid;
s) 4-[(heptyloxy)carbonyl]phenyl 5-bromopyridine-3-carboxylate;
t) (9Z,12Z,15Z)-octadeca-9,12,15-trienoic acid;
u) (4Z,7Z,10Z,13Z,16Z,19Z)-docosa-4,7,10,13,16,19-hexaenoic acid;
v) (9Z,12R)-12-hydroxyoctadec-9-enoic acid;
w) (9Z)-octadec-9-enoic acid;
x) 4-[(1-benzofuran-2-yl)formamido]-N-[2-(1H-indol-3-yl)ethyl]butanamide; and
y) 4-(methylsulfanyl)phenyl 4-heptylbenzoate.

In the present invention, the compound may specifically bind to the spike protein of the coronavirus.

In the present invention, the compounds a) to y) are known natural products and the molecular structures thereof may be easily found and obtained through a database. Specifically, the names of the compounds a) to y) are determined based on the IUPAC nomenclature and the MolPort ID of each compound disclosed in MolPort (https:/www.molport.com/shop/index), which is a typical compound database and merchant, is shown in Table 1 below.

TABLE 1

| | Compound MolPort ID | Molecular formula | IUPAC |
|---|---|---|---|
| a | MolPort-009-758-577 | C27H45NO6 | [3-(2,2-dimethyloxan-4-yl)- |
| | | | 6-methylheptyl]]({[4-(propan- |
| | | | 2-yloxy) phenyl]]methyl}) amine; |
| | | | oxalic acid |
| b | MolPort-005-980-235 | C26H32N2O2 | 10,10-dimethyl-10a-[(1E)-2- |
| | | | [3-(pentyloxy) phenyl]]ethenyl]]- |
| | | | 3H,4H,10H,10aH-pyrimido[1, |
| | | | 2-a]indol-2-ol |
| c | MolPort-000-829-850 | C27H30N2O6 | 3-{1-[3-(3-phenoxyphenoxy) |
| | | | propyl]]piperidin-2-yl} |
| | | | pyridine and oxalic acid |
| d | MolPort-027-564-475 | C27H33N3O2 | N-{[(1S,9aR)-octahydro-1H- |
| | | | quinolizin-1-yl]methyl}-2- |
| | | | [6-(benzyloxy)-1H-indol-1- |
| | | | yllacetamide |
| e | MolPort-027-564-039 | C26H28N2O3 | N-{[(1S,9aR)-octahydro-1H- |
| | | | quinolizin-1-yl]methyl}-3- |
| | | | (2-oxo-2H-chromen-3- |
| | | | yl) benzamide |
| f | MolPort-002-151-168 | C25H35NO3 | 3-(3,5-di-tert-butyl-4- |
| | | | hydroxyphenyl)propyl 2- |
| | | | (phenylamino)acetate |
| g | MolPort-005-947-745 | C25H28FN3O3 | N-[(4-fluorophenyl)methyl]- |
| | | | 2-[(3R,4S)-3-{[5- |
| | | | (phenoxymethyl)-1,2-oxazol- |
| | | | 3-yl]methyl}piperidin-4- |
| | | | yllacetamide |
| h | MolPort-003-711-757 | C20H23NO4 | 4-[(heptyloxy) carbonyl]]phenyl |
| | | | pyridine-3-carboxylate |
| i | MolPort-002-515-990 | C20H3O5 | 2-octyl-1,3-dioxan-5-yl |
| | | | 4-methoxybenzoate |
| j | MolPort-002-526-760 | C28H25N3O | N,1-bis(2-phenylethyl)-9H- |
| | | | pyrido[3,4-b]indole-3- |
| | | | carboxamide |
| k | MolPort-008-266-638 | C22H32O2 | (4Z,7E,10E,13E,16E,19E)- |
| | | | docosa-4,7,10,13,16,19- |
| | | | hexaenoic acid |
| l | MolPort-002-520-573 | C23H29NO3S | [(1R,9aR)-octahydro-1H- |
| | | | quinolizin-1-yl]methyl 5- |
| | | | [(benzylsulfanyl)methyl]] |
| | | | furan-2-carboxylate |
| m | MolPort-002-526-563 | C25H25NO6 | 4-oxo-8-propyl-1H,2H,3H,4H- |
| | | | cyclopenta[c]chromen-7-yl 2- |
| | | | {[(benzyloxy)carbonyl]]amino} |
| | | | acetate |
| n | MolPort-003-939-688 | C23H38O2 | tricosa-10,12-diynoic acid |
| o | MolPort-000-424-809 | C23H24O6 | ethyl 2-tert-butyl-5-(4- |
| | | | methoxybenzoyloxy)-1- |
| | | | benzofuran-3-carboxylate |
| P | MolPort-002-152- | C25H41NO3 | 3-(3,5-di-tert-butyl-4- |
| | 403 | | hydroxyphenyl) propyl 2- |
| | | | (cyclohexylamino) acetate |
| q | MolPort-003-933-180 | C20H30O2 | (5Z,8Z,11Z,14Z,17Z)-icosa- |
| | | | 5,8,11,14,17-pentaenoic acid |
| r | MolPort-003-709-991 | C21H24O4 | 4-(4-heptylbenzoyloxy) benzoic |
| | | | acid |
| S | MolPort-003-713-431 | C20H22BrNO4 | 4-[(heptyloxy) carbonyl]]phenyl |
| | | | 5-bromopyridine-3-carboxylate |
| t | MolPort-003-937-817 | C18H30O2 | (9Z,12Z,15Z)-octadeca- |
| | | | 9,12,15-trienoic acid |
| u | MolPort-003-932-890 | C22H32O2 | (4Z,7Z,10Z,13Z,16Z,19Z)- |
| | | | docosa-4,7,10,13,16,19- |
| | | | hexaenoic acid |
| v | MolPort-003-939-239 | C18H34O3 | (9Z,12R)-12-hydroxyoctadec- |
| | | | 9-enoic acid |
| w | MolPort-001-788-274 | C18H34O2 | (9Z)-octadec-9-enoic acid |
| x | MolPort-005-974- | C23H23N3O3 | 4-[(1-benzofuran-2- |
| | 761 | | yl) formamido]]-N-[2-(1H- |
| | | | indol-3-yl) ethyl]]butanamide |
| y | MolPort-003-713-147 | C21H26O2S | 4-(methylsulfanyl) phenyl 4- |
| | | | heptylbenzoate |

In the present invention, the compound is preferably selected from the following compounds:
b) 10,10-dimethyl-10a-[(1E)-2-[3-(pentyloxy)phenyl]ethenyl]-3H,4H,10H,10aH-pyrimido[1,2-a]indol-2-ol;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
i) 2-octyl-1,3-dioxan-5-yl 4-methoxybenzoate;
n) Tricosa-10,12-dienoic acid;
o) Ethyl 2-tert-butyl-5-(4-methoxybenzoyloxy)-1-benzofuran-3-carboxylate; and
r) 4-(4-heptylbenzoyloxy)benzoic acid.

In the present invention, the compound is, most preferably, b) 10,10-dimethyl-10a-[(1E)-2-[3-(pentyloxy)phenyl]ethenyl]-3H,4H,10H,10aH-pyrimido[1,2-a]indol-2-ol.

In the present invention, the composition may contain a derivative of the compound or a salt thereof.

As used herein, the term “derivative” refers to a compound having a structure that is sufficiently similar to the structure of a compound disclosed herein, a compound that those skilled in the art would expect to have the same or similar activity and use as the claimed compound, or a structure derived from the structure of a parent compound (e.g., a compound described herein) that would be expected to induce the same or similar activity and use as the claimed compound as a precursor, based on the similarity thereof. Examples of derivatives include salts, isomers, esters, amides of the parent compound, salts of esters or amides, and N-oxides.

As used herein, the term “salt” refers to an addition salt of an inorganic acid salt, an organic acid salt, or a metal salt of the compound, and may be a “cosmetically acceptable salt” when used in a cosmetic composition, a “sitologically acceptable salt” when used in a food composition, or a “pharmaceutically acceptable salt” when used in a pharmaceutical composition. The pharmaceutically acceptable salt may be a salt that does not cause serious irritation to an organism to which the compound is administered and does not impair the biological activity and physical properties of the compound. As used herein, the term “pharmaceutically acceptable salt” refers to a formulation of a compound which does not cause serious irritation to an organism to which the compound is administered and does not impair the biological activity and physical properties of the compound. The pharmaceutically acceptable salt includes an acid addition salt formed by an acid which forms a non-toxic acid addition salt containing a pharmaceutically acceptable anion, for example, an inorganic acid such as hydrochloric acid, sulfuric acid, nitric acid, phosphoric acid, hydrobromic acid, hydroiodic acid, etc., an organic carboxylic acid such as tartaric acid, formic acid, citric acid, acetic acid, trichloroacetic acid, trifluoroacetic acid, gluconic acid, benzoic acid, lactic acid, fumaric acid, maleic acid, salicylic acid, etc., a sulfonic acid such as methanesulfonic acid, ethanesulfonic acid, benzenesulfonic acid, p-toluenesulfonic acid, etc. Examples of the pharmaceutically acceptable carboxylic acid salts include: metal salts or alkaline earth metal salts formed by lithium, sodium, potassium, calcium, magnesium, and the like; amino acid salts such as lysine, arginine, guanidine, and the like; organic salts such as dicyclohexylamine, N-methyl-D-glucamine, tris(hydroxymethyl)methylamine, diethanolamine, choline, and triethylamine, and the like. Acids such as oxalic acid, although not pharmaceutically acceptable, may be used in the preparation of useful salts as intermediates for obtaining the compounds of the present invention and pharmaceutically acceptable salts thereof.

In the present invention, the antiviral composition exhibits antiviral activity against coronavirus or influenza virus.

As used herein, the term “anti-virus” or “anti-viral activity/(efficacy)” means inhibiting or shielding the activity of a virus, such as virus entry (infection) into cells, virus proliferation, and virus toxicity. In the present invention, the anti-virus preferably means the ability to inhibit virus particles from adhering to the cell membrane (or cell membrane protein) of a host cell and entering the cell. This anti-virus ability interferes with the mechanism of the virus necessary for replicating or propagating virus particles using the host cells and enables the compound contained as an active ingredient in the composition of the present invention to exhibit anti-viral activity.

In the present invention, the anti-viral composition inhibits the activity of a coronavirus and/or influenza virus and preferably has the ability to inhibit the cell entry (infection), replication, and/or proliferation of a coronavirus and/or influenza virus, but is not limited thereto.

In the present invention, the compound specifically binds to the spike protein of a coronavirus.

As used herein, the term “spike protein” refers to a protein that binds to the ACE2 receptor of a host cell on the surface of SARS-CoV-2 and participates in intracellular entry. It has been reported that, when the protein binds to an antibody, peptide, low molecular weight substance, or the like, thereby interfering with binding to ACE2, intracellular entry, replication, and/or proliferation of the virus can be inhibited.

Therefore, it is obvious to those skilled in the art that the antiviral composition of the present invention exhibits antiviral activity against viruses expressing proteins having substantially the same function and sequence as the spike protein in addition to the coronavirus described above.

In the present invention, the coronavirus is preferably SARS-CoV-2 virus, and more preferably, SARS-CoV-2 virus.

In the present invention, as another example, the coronavirus may be, but is not limited to, Ancestral SARS-CoV-2 (lineage A), Alpha (B.1.1.7_UK), Beta (B.1.351_South Africa), Gamma (P.1_Brazil), Delta (B.1.617.2_India), or Omicron (B.1.1.529) variant coronavirus.

As used herein, the term “influenza virus” refers to an influenza virus of the Orthomyxoviridae family. For example, the influenza virus may be classified into influenza A/B/C viruses, and in the present invention, the influenza virus may be an influenza A virus, an influenza B virus, or an influenza C virus, and is preferably an influenza A virus or an influenza B virus, but is not limited thereto.

In the present invention, the influenza virus encompasses subtypes and for example, the influenza A virus may be classified into 18 subtypes and 11 subtypes, respectively, according to the H serotype or N serotype, but is not limited thereto.

In the present invention, the coronavirus and influenza virus may include viruses, subtypes, and mutant viruses that are classified as coronavirus or influenza virus, or are derived therefrom.

The compound contained in the antiviral composition of the present invention is a natural product or a natural product derivative, which is less toxic than conventional synthetic drugs, and has already been empirically proven to be safe and effective when consumed as food, and may be produced and used in the form of various products in addition to pharmaceutical compositions.

The antiviral composition may further contain appropriate carriers, excipients, and diluents commonly used in the field to which the composition is applied, and may be prepared and used in the form of a pharmaceutical composition administered to a vertebrate, preferably a mammal including a human, a washing/cleaning composition for tableware, kitchenware, household goods, and other various objects, a disinfecting composition, a cosmetic composition, a food composition, or a pharmaceutical composition, but is not limited thereto, and may be prepared in all forms commonly known as conventional antiviral agents.

In the present invention, the washing/cleaning composition is used for washing and/or cleaning the body or various objects and may further contain a natural preservative (for example, an antioxidant such as tocopherol, ascorbic acid, ascorbyl palmitate, gallic acid, quercetin, flavonol, flavonoid, ethoxyquin, lignan glycosides, and mixtures thereof), a stabilizer (for example, β-cyclodextrin, citric acid, magnesium chloride (MgCl2), ethylenediaminetertraacetic acid (EDTA salt), and dibutyl hydroxy toluene (BHT), etc.), or a fragrance, in addition to the compound as an active ingredient, but is not limited thereto.

In the present invention, the disinfecting composition refers to a composition capable of preventing and/or inhibiting virus infection. In a comprehensive sense, the term “disinfection” as used herein encompasses reducing or eliminating the toxicity of a virus in addition to killing a virus or bacteria.

In the present invention, the disinfecting composition may be formulated in the form of a solid, powder, liquid, gel, emulsion, or aerosol, and may also be prepared as a solid such as a commercially available soap by further mixing with a natural additive that undergoes a saponification reaction, but is not limited thereto.

In the present invention, the cosmetic composition may be used as an additive for cosmetics, depending on the antiviral efficacy of the compound. In the present invention, the cosmetic composition may further contain a cosmetically acceptable excipient or carrier, and for example, depending on the formulation, may further contain various known additives, and for example, may further contain an additive selected from the group consisting of a carrier, an emulsifier, a moisturizer, a surfactant, a chelating agent, an antioxidant, a bactericide, a stabilizer, and any combination thereof.

In the present invention, the cosmetic composition may be prepared and used in various known formulations depending on the intended use and method of use. For example, the cosmetic composition according to the present invention may be in the form of, but is not limited to, a toner, a facial lotion, a body lotion, a nourishing cream, a moisturizing cream, an eye cream, an essence, a cosmetic ointment, a spray, a gel, a pack, a sunscreen, a makeup base, a foundation, a powder, a cleansing cream, a cleansing lotion, a cleansing oil, a cleansing foam, a soap, or a body wash.

In the present invention, the food composition may contain the compound as an active ingredient and thus exhibit an antiviral effect, and may be used for the prevention or amelioration of coronavirus or influenza infection.

As used herein, the term “food composition” is used in a broad sense to encompass substances containing nutrients, and examples of the food composition include meat, sausage, bread, chocolate, candy, snacks, confectionery, pizza, ramen, other noodles, gum, dairy products including ice cream, various soups, beverages, tea, drinks, alcoholic beverages, vitamin complexes, prebiotics, probiotics, postbiotics, health supplement food, health functional food, health food and the like. The term “food composition” is used in an ordinary sense to encompass “food” and “food additive” or “composition for food additive” added to food.

In the present invention, when the food is provided as feed for animals other than humans, it has substantially the same meaning as and may be used interchangeably with “feed” and therefore, in the present invention, the food composition may refer to a feed composition, a feed additive (feed additive composition).

As used herein, the term “functional food” has the same meaning as the term “food for special health use (FoSHU)” and refers to a food having strong medical and pharmaceutical effects that has been processed to efficiently provide a bioregulatory function as well as a nutrition supply function. Here, the term “functional” means obtaining beneficial effects for health purposes, such as controlling nutrients or exhibiting physiological effects with regard to the structures and functions of the human body. The food of the present invention may be prepared by a method commonly used in the art, and the preparation may be performed using raw materials and ingredients commonly added in the art. In addition, the food may also be prepared into any formulation recognized as a food without limitation, and the health functional food according to the present invention may be in the form of a powder, granule, tablet, capsule, or beverage.

The term “health food” refers to a food having an active health maintenance or promotion effect beyond that of a general food, and the term “health supplement food” refers to a food ingested for the purpose of health supplement. In some cases, the terms “health functional food”, “health food”, and “health supplement food” are used interchangeably.

The food composition may further contain a physiologically acceptable carrier, and there is no particular limitation as to the kind of carrier, and any carrier commonly used in the art may be used.

In addition, the composition may contain additional ingredients that are commonly used in food compositions to improve smell, taste, visual quality (appearance), and the like. For example, the composition may contain vitamins A, C, D, E, B1, B2, B6 and B12, niacin, biotin, folate, pantothenic acid, and the like. In addition, the composition may contain minerals such as zinc (Zn), iron (Fe), calcium (Ca), chromium (Cr), magnesium (Mg), manganese (Mn), copper (Cu), and chromium (Cr). In addition, the composition may contain amino acids such as lysine, tryptophan, cysteine, and valine.

In addition, the composition may contain food additives such as preservatives (potassium sorbate, sodium benzoate, salicylic acid, sodium dehydroacetate, etc.), disinfectants (bleached powder and highly bleached powder, sodium hypochlorite, etc.), antioxidants (butylhydroxyanisole (BHA), butylhydroxytoluene (BHT), etc.), colorants (tar color, etc.), color-developing agents (sodium nitrite, sodium nitrite, etc.), bleaching agents (sodium sulfite), seasonings (MSG, etc.), sweeteners (dulcin, cyclamate, saccharin, sodium, etc.), flavorings (vanillin, lactones, etc.), expanding agents (alum, D-potassium hydrogen tartrate, etc.), reinforcing agents, emulsifiers, thickeners, coating agents, gum base agents, anti-foaming agents, solvents, and enhancers. The additive may be selected according to the type of food and used in an appropriate amount.

The composition according to the present invention may be used at an effective dose. For example, the composition may be consumed at a food composition sitologically effective dose. In the present invention, the term “effective dose,” “sitologically effective dose,” and “pharmaceutically effective amount” mean an amount sufficient to treat a disease at a reasonable benefit/risk ratio applicable to the object of the present invention, which is the prevention or treatment of the disease, and the effective dose level may be determined depending on the type and severity of the disease of the subject, the activity of the drug, the sensitivity to the drug, the time of administration, the route of administration, and the excretion rate, the treatment period, the concurrently used drugs, and other factors well known in the pharmaceutical field.

In the present invention, the antiviral composition may be prepared in the form of a pharmaceutical composition and the details thereof are described in detail with reference to the pharmaceutical composition for preventing or treating coronavirus infection or influenza.

In another aspect, the present invention is directed to antiviral use of the compound against coronavirus or influenza virus.

In another aspect, the present invention is directed to the use of the compound for the preparation of an anti-viral composition against coronavirus or influenza virus.

In another aspect, the present invention is directed to a method for inhibiting infection, activity and/or proliferation of coronavirus or influenza virus, including treatment with the compound.

In one embodiment of the present invention, it was found that the compound of the present invention can effectively inhibit intracellular entry (infection), replication and proliferation of SARS-CoV-2 virus, and it was further found that the compound can effectively inhibit replication and proliferation of influenza virus.

Therefore, the present invention provides a pharmaceutical composition for preventing or treating coronavirus infection or influenza, containing a compound selected from the group consisting of the following compounds, a derivative thereof or a pharmaceutically acceptable salt thereof as an active ingredient:
a) [3-(2,2-dimethyloxan-4-yl)-6-methylheptyl]({[4-(propan-2-yloxy)phenyl]methyl})amine and oxalic acid;
b) 10,10-dimethyl-10a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
g) N-[(4-fluorophenyl)methyl]-2-[(3R,4S)-3-{[5-(phenoxymethyl)-1,2-oxazol-3-yl]methyl}piperidin-4-yl]acetamide;
h) 4-[(heptyloxy)carbonyl]phenyl pyridine-3-carboxylate;
i) 2-octyl-1,3-dioxan-5-yl 4-methoxybenzoate;
j) N,1-bis(2-phenylethyl)-9H-pyrido[3,4-b]indole-3-carboxamide;
k) (4Z,7E,10E,13E,16E,19E)-docosa-4,7,10,13,16,19-hexaenoic acid;
l) [(1R,9aR)-octahydro-1H-quinolizin-1-yl]methyl 5-[(benzylsulfanyl)methyl]furan-2-carboxylate;
m) 4-oxo-8-propyl-1H,2H,3H,4H-cyclopenta[c]chromen-7-yl 2-{[(benzyloxy)carbonyl]amino}acetate;
n) Tricosa-10,12-dienoic acid;
o) Ethyl 2-tert-butyl-5-(4-methoxybenzoyloxy)-1-benzofuran-3-carboxylate;
p) 3-(3,5-di-tert-butyl-4-hydroxyphenyl)propyl 2-(cyclohexylamino)acetate;
q) (5Z,8Z,11Z,14Z,17Z)-icosa-5,8,11,14,17-pentaenoic acid;
r) 4-(4-heptylbenzoyloxy)benzoic acid;
s) 4-[(heptyloxy)carbonyl]phenyl 5-bromopyridine-3-carboxylate;
t) (9Z,12Z,15Z)-octadeca-9,12,15-trienoic acid;
u) (4Z,7Z,10Z,13Z,16Z,19Z)-docosa-4,7,10,13,16,19-hexaenoic acid;
v) (9Z,12R)-12-hydroxyoctadec-9-enoic acid;
w) (9Z)-octadec-9-enoic acid;
x) 4-[(1-benzofuran-2-yl)formamido]-N-[2-(1H-indol-3-yl)ethyl]butanamide; and
y) 4-(methylsulfanyl)phenyl 4-heptylbenzoate.

In the present invention, unless otherwise specifically mentioned, the detailed description of the compound may share the same features as those described with reference to the antiviral composition of the present invention.

In the present invention, the term “coronavirus infection” or “coronavirus disease” means that a human or animal has cells infected with a coronavirus. The infection may be established, in particular, by detecting and/or performing a virus titration from a respiratory sample, or by assaying blood-circulating coronavirus-specific antibodies.

As used herein, the term “influenza” means that a human or animal organism has cells infected with an influenza virus. The infection may be established, in particular, by detecting and/or performing virus titration from a respiratory sample, or by assaying blood-circulating influenza virus-specific antibodies.

The detection in a subject infected with such a specific virus may be performed by conventional diagnostic methods (PCR) well known to those skilled in the art, in particular in molecular biology.

In the present invention, the coronavirus is preferably SARS coronavirus, and more preferably SARS-CoV-2 virus.

In the present invention, as another example, the coronavirus may be Ancestral SARS-CoV-2 (lineage A), Alpha (B.1.1.7_UK), Beta (B.1.351_South Africa), Gamma (P.1_Brazil), Delta (B.1.617.2_India), or Omicron (B.1.1.529) mutant coronavirus, but is not limited thereto.

As used herein, the term “influenza virus” refers to an influenza virus of the Orthomyxoviridae family. For example, the influenza virus may be classified into influenza A/B/C viruses, and in the present invention, the influenza virus may be an influenza A virus, an influenza B virus, or an influenza C virus, and is preferably an influenza A virus or an influenza B virus, but is not limited thereto.

In the present invention, the influenza virus is used to mean including subtypes, and for example, the influenza A virus may be classified into 18 subtypes and 11 subtypes, respectively, according to the H serotype or N serotype, but is not limited thereto.

In the present invention, the coronavirus and influenza virus are used as concepts including viruses, subtypes, and mutant viruses belonging to coronavirus or influenza virus in terms of classification, or derived therefrom.

As used herein, the terms “prevention” and “treatment” should be interpreted in the broadest sense, and the term “prevention” refers to prevention of the progression of one or more of the clinical symptoms of a disease in a patient who may be exposed to or susceptible to the disease but has not yet experienced or manifested symptoms of the disease. The term “treatment” means any action that inhibits or reduces the development of a disease or one or more of clinical symptoms thereof.

As used herein, the term “treatment” means combating SARS-CoV-2 infection in a human or animal. By administering at least one composition according to the present invention, the viral infection rate (infection titer) in the organism will be reduced, and preferably, the virus may be completely eliminated from the organism. As used herein, the term “treatment” also means ameliorating symptoms associated with the viral infection (respiratory syndrome, renal failure, fever).

The pharmaceutical composition for preventing or treating coronavirus infection or influenza of the present invention may contain a pharmaceutically acceptable carrier and may be prepared along with the carrier.

As used herein, the term “pharmaceutically acceptable carrier” refers to a carrier or diluent that does not impair the biological activities or properties of the administered compound without irritating an organism. The term “pharmaceutically acceptable carrier” as used herein refers to a carrier or diluent that does not impair the biological activities or properties of the administered compound and does not stimulate an organism. Pharmaceutically acceptable carriers for compositions, which are formulated into liquid solutions, are sterilized and biocompatible, and examples thereof include saline, sterile water, buffered saline, albumin injection solutions, dextrose solutions, maltodextrin solutions, glycerol and mixtures thereof. If necessary, other conventional additives such as antioxidants, buffers and bacteriostatic agents may be added. In addition, diluents, dispersants, surfactants, binders and lubricants may be further added to formulate injectable solutions such as aqueous solutions, suspensions and emulsions, pills, capsules, granules, or tablets.

The composition for preventing or treating coronavirus infection or influenza containing the compound and the pharmaceutically acceptable carrier may be applied to any formulation containing the same as an active ingredient and may be prepared for oral or parenteral formulation. The pharmaceutical formulation may include formulations suitable for oral, rectal, nasal, topical (including under the cheek and tongue), subcutaneous, vaginal or parenteral (including intramuscular, subcutaneous and intravenous) administration, or inhalation or insufflation.

Examples of formulations for oral administration containing the pharmaceutical composition of the present invention as an active ingredient include tablets, troches, lozenges, aqueous or oily suspensions, prepared powders or granules, emulsions, hard or soft capsules, syrups or elixirs. In order to prepare formulations such as tablets and capsules, a binder such as lactose, saccharose, sorbitol, mannitol, starch, amylopectin, cellulose or gelatin, an excipient such as dicalcium phosphate, a disintegrant such as corn starch or sweet potato starch, a lubricant such as calcium stearate, sodium stearyl fumarate or polyethyleneglycol wax may be incorporated, and capsule formulations may further contain a liquid carrier such as a fatty oil, in addition to the above-mentioned ingredients.

The pharmaceutical composition of the present invention containing an active ingredient is preferably administered intranasally or intranasally and the pharmaceutical composition of the present invention may be formulated as a spray or aerosol or as a formulation for inhalation. The pharmaceutical formulation for intranasal or intranasal administration is prepared according to techniques well-known in the pharmaceutical field as a solution in saline using benzyl alcohol or other suitable preservatives, absorption accelerators to enhance bioavailability, fluorocarbons and/or other solubilizers or dispersants known in the art.

In another embodiment, the composition for treating coronavirus infection or influenza of the present invention may be a sterile injectable formulation such as a sterile injectable aqueous or oily suspension. This suspension may be formulated according to techniques known in the art using suitable dispersing or wetting agents (e.g. Tween 80) and suspending agents. The sterile injectable formulation may also be a sterile injectable solution or suspension in a non-toxic parenterally acceptable diluent or solvent (e.g., a solution in 1,3-butanediol). Pharmaceutically acceptable vehicles and solvents include mannitol, water, Ringer's solution and isotonic sodium chloride solution. In addition, sterile nonvolatile oils are commonly used as solvents or suspension media. For this purpose, any less irritating nonvolatile oil including synthetic mono- or diglycerides may be used. Fatty acids such as oleic acid and glyceride derivatives thereof, like pharmaceutically acceptable natural oils (e.g. olive oil or castor oil), particularly polyoxyethylated natural oils thereof, are useful for injectable formulations.

In another aspect, the present invention is directed to a method of preventing or treating an immune disease including administering the compound or the pharmaceutical composition to a subject.

As used herein, the term “administering” means introducing the pharmaceutical composition of the present invention into a patient by any suitable method. The composition of the present invention may be administered through various oral or parenteral routes, as long as it may reach the target tissue. Specifically, in addition to oral administration through the mouth, the pharmaceutical composition may be administered through parenteral routes such as intranasal or intranasal administration. As another example of parenteral administration, the pharmaceutical composition may be administered in a conventional manner via an inhalation, oral, rectal, topical, intravenous, intraperitoneal, intramuscular, intraarterial, transdermal, intranasal, ocular or intradermal route.

The treatment method of the present invention includes administering a pharmaceutically effective amount of the composition according to the present invention. It will be obvious to those skilled in the art that an appropriate total daily dose can be determined by a physician within the scope of correct medical judgment. The specific therapeutically effective amount for a certain patient preferably depends upon a variety of factors including the type and extent of the response to be achieved, as well as the presence of other agents used, the specific composition, and age, body weight, general health conditions, gender and diet of the patient, administration time, administration route and the secretion rate of the composition, treatment period, and drugs used in conjunction with or concurrently with the specific composition, and upon similar factors well-known in the field of medicine. Therefore, the effective amount of the composition for treating coronavirus disease-2019, which is suitable for the object of the present invention, is preferably determined in consideration of the aforementioned factors.

In addition, the treatment method of the present invention may be applied to any animals that may be infected with coronavirus or influenza virus, and the animals include humans and primates as well as livestock such as cows, pigs, sheep, horses, dogs and cats.

It is obvious that the composition for use according to the invention contains the above-described compound, and may also contain another active compound in addition to suitable pharmaceutically acceptable carriers. The active compound may be a compound enabling the activity of these compounds to be improved, or even other active agents known for a particular activity. These further active compounds may be selected from the pharmaceutical classes of agents mentioned in application WO 2015/157223, namely from the antibacterial agents, antiparasitic agents, neurotransmission inhibitors, estrogen receptor inhibitors, DNA synthesis and replication inhibitors, protein maturation inhibitors, kinase pathway inhibitors, cytoskeleton inhibitors, lipid metabolism inhibitors, anti-inflammatory agents, ion channel inhibitors, apoptosis inhibitors and cathepsin inhibitors. These active compounds may be selected in particular from antibacterial agents, ion channel inhibitors, immunosuppressive agents and antiviral agents. As an antiviral agent, acyclovir can in particular be mentioned.

The pharmaceutical composition for treating SARS-CoV-2 infection of the present invention may contain at least another antiviral agent in addition to the above compound. It is understood that this antiviral agent will be used at doses necessary to have antiviral action, this dose being designated as being “efficient”, this dosage being possibly readily determined by those skilled in the art. For the purposes of the present invention, an antiviral agent designates a compound acting on a virus, by inhibiting and/or slowing and/or preventing the associated viral infection.

Antiviral agents are categorized based on mode of action. These antiviral agents include, in particular:
nucleotide analogues, which interfere with or stop DNA or RNA synthesis; as well as inhibitors of the enzymes involved in DNA or RNA synthesis (helicase, replicase);
compounds which inhibit the virus maturation steps during its replication cycle;
compounds which interfere with cell membrane binding, or virus entry into host cells (fusion or entry inhibitors);
agents which prevent the virus from being expressed within the host cell after entry, by blocking disassembly within the cell;
agents which restrict virus propagation to other cells.

One can mention in particular antiviral agents well known to those skilled in the art, intended to fight RNA viruses, such as protease inhibitors, helicase inhibitors, and SARS-CoV-2 virus cell entry inhibitors in target cells.

In another aspect, the present invention is directed to a method of treating coronavirus disease 2019 using the composition.

In another aspect, the present invention is directed to the use of the composition for the treatment of coronavirus disease 2019.

### EXAMPLES

Hereinafter, the present invention will be described in more detail with reference to examples. It will be obvious to skilled in the art that these examples serve merely to illustrate the present invention, and the scope of the present invention is not limited by these examples.

### Example 1: Construction of Compound Library for Drug Virtual Screening

In order to construct a compound library including natural products or natural product derivatives, 366,096 compounds were collected without duplication using MolPort, Super Natural II, MIBiG, and PubChem databases. All collected compounds were expressed in SMILES and salt removal, duplication, and structural abnormalities thereof were identified. All operations dealing with compound structures was performed with Python scripts using the open source RDKit package.

### Example 2: Acquisition of 3D Structure of Viral Drug Target for Protein-Ligand Docking

### 2-1: Structure of SARS-CoV-2 Spike Protein

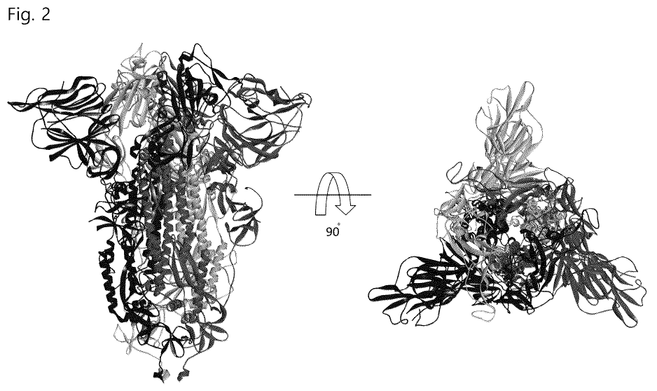

Several 3D structures of the spike protein of SARS-CoV-2 are stored in the Protein Data Bank database, and thereamong, structures having an excellent crystal structure resolution of 2.6 Å and a pocket in the receptor-binding domain capable of binding to drugs were selected (FIG. 2, PDB ID: 6ZGE).

### Example 3: Prediction of SARS-CoV-2 Spike Inhibitors Through Virtual Drug Screening

### Example 3-1: Screening Using Ligand Shape Similarity

Considering the shape and size of the pocket present in the receptor-binding domain of SARS-CoV-2 spike, a mock ligand composed of a carbon chain was formed and docking simulations were performed on the pocket. A three-dimensional structure of the mock ligand that was optimized for the pocket shape was obtained and the ligand shape similarity was compared between the compounds of the compound library obtained in Example 1 and these active compounds as a reference structure. Since the compound library obtained in Example 1 is stored as a two-dimensional structure rather than a three-dimensional structure, conversion into the three-dimensional structure was performed. Using the experimental-torsion basic knowledge distance geometry algorithm contained in the RDKit package, up to 100 three-dimensional conformers with the highest probability were experimentally generated for each compound. A total of 36,609,600 three-dimensional conformers was generated from 366,096 compounds. Calculation of the ligand shape similarity between these conformers and known active ligands was performed. Calculation of the ligand shape similarity was performed using USR (Ultrafast Shape Recognition). After USR similarity was calculated, the next step, docking simulation analysis, was performed on 6,070 compounds having a value of 0.75 or more.

To predict the binding behavior and binding intensity between the protein and each compound, molecular docking simulation was performed using AutoDock Vina. A PDBQT file, an input file of the AutoDock Vina program, was created for each of the protein and the ligand using OpenBabel and MGLTools packages. A grid box was set from the center of the binding site in the receptor-binding domain of the Spike protein (10 Å *10 Å *10 Å). 26 compounds with a docking binding energy value of −7.0 kcal/mol or less were selected (Table 2).

TABLE 2

| | | Molecular | |
|---|---|---|---|
| Compound ID | Docking energy (kcal/mol) | formula | IUPAC |
| MolPort-002-520-573 | −9.6 | C23H29NO3S | [(1R,9aR)- |
| | | | octahydro- |
| | | | 1H-quinolizin- |
| | | | 1-yl]methyl |
| | | | 5-[(benzylsul |
| | | | fanyl) methyl] |
| | | | furan-2- |
| | | | carboxylate |
| MolPort-027-564-475 | −9.3 | C27H33N3O2 | N-{[(1S,9aR)- |
| | | | octahydro-1H- |
| | | | quinolizin-1- |
| | | | yl]methyl}- |
| | | | 2-[6-(benzyloxy)- |
| | | | 1H-indol-1- |
| | | | yllacetamide |
| MolPort-000-829-850 | −9.3 | C27H30N2O6 | 3-{1-[3-(3- |
| | | | phenoxyphen |
| | | | oxy) propyl] |
| | | | piperidin-2-yl} |
| | | | pyridine and oxalic |
| | | | acid |
| MolPort-002-526-760 | −9.3 | C28H25N3O | N,1-bis(2- |
| | | | phenylethyl)-9H- |
| | | | pyrido [3,4-b] |
| | | | indole-3- |
| | | | carboxamide |
| MolPort-008-266-638 | −9.2 | C22H32O2 | (4Z,7E,10E, |
| | | | 13E,16E,19E)- |
| | | | docosa-4,7,10,13,16, |
| | | | 19-hexaenoic acid |
| MolPort-003-932-890 | −9.2 | C22H32O2 | (4Z,7Z,10Z, |
| | | | 13Z,16Z,19Z)- |
| | | | docosa-4,7,10,13,16, |
| | | | 19-hexaenoic acid |
| MolPort-005-947-745 | −9.1 | C25H28FN3O3 | N-[(4-fluorophenyl) |
| | | | methyl]]-2-[(3R,4S)- |
| | | | 3-{[5-(phenoxymethyl)- |
| | | | 1,2-oxazol-3-yl]methyl} |
| | | | piperidin-4-yl]acetamide |
| MolPort-000-424-809 | −9.1 | C23H24O6 | ethyl 2-tert-butyl- |
| | | | 5-(4-methoxybenzoyloxy)- |
| | | | 1-benzofuran-3- |
| | | | carboxylate |
| MolPort-005-974-761 | −9.0 | C23H23N3O3 | 4-[(1-benzofuran- |
| | | | 2-yl) formamido]-N-[2- |
| | | | (1H-indol-3-yl) |
| | | | ethyl]] butanamide |
| MolPort-003-709-991 | −8.8 | C21H24O4 | 4-(4-heptylbenzoyloxy) |
| | | | benzoic acid |
| MolPort-005-980-235 | −8.8 | C26H32N2O2 | 10,10-dimethyl-10a-[(1E)- |
| | | | 2-[3-(pentyloxy) |
| | | | phenyllethenyl]]- |
| | | | 3H,4H,10H,10aH- |
| | | | pyrimido[1,2-a]indol- |
| | | | 2-o1 |
| MolPort-003-933-180 | −8.7 | C20H30O2 | (5Z,8Z,11Z, |
| | | | 14Z,17Z)-icosa- |
| | | | 5,8,11,14,17-pentaenoic |
| | | | acid |
| MolPort-002-151-168 | −8.7 | C25H35NO3 | 3-(3,5-di-tert-butyl-4- |
| | | | hydroxyphenyl) |
| | | | propyl 2-(phenylamino) |
| | | | acetate |
| MolPort-002-526-563 | −8.5 | C25H25NO6 | 4-oxo-8-propyl- |
| | | | 1H,2H,3H,4H |
| | | | cyclopenta[c]]chromen- |
| | | | 7-yl 2-{[(benzyloxy) |
| | | | carbonyl]amino}acetate |
| MolPort-003-711-757 | −8.4 | C20H23NO4 | 4-[(heptyloxy) |
| | | | carbonyl]phenyl |
| | | | pyridine-3-carboxylate |
| MolPort-002-515-990 | −8.4 | C20H30O5 | 2-octyl-1,3-dioxan- |
| | | | 5-yl 4-methoxybenzoate |
| MolPort-002-152-403 | −8.3 | C25H41NO3 | 3-(3,5-di-tert-butyl-4- |
| | | | hydroxyphenyl) propyl |
| | | | 2-(cyclohexylamino) acetate |
| MolPort-003-713-431 | −8.3 | C20H22BrNO4 | 4-[(heptyloxy) |
| | | | carbonyl]phenyl 5- |
| | | | bromopyridine-3- |
| | | | carboxylate |
| MolPort-027-564-039 | −8.3 | C26H28N2O3 | N-{[(1S,9aR)- |
| | | | octahydro-1H- |
| | | | quinolizin-1-yl]methyl}- |
| | | | 3-(2-0x0-2H-chromen-3-yl) |
| | | | benzamide |
| MolPort-003-939-688 | −8.3 | C23H38O2 | tricosa-10,12-diynoic |
| | | | acid |
| MolPort-003-713-147 | −8.2 | C21H26O2S | 4-(methylsulfanyl) |
| | | | phenyl 4-heptylbenzoate |
| MolPort-003-937-817 | −8.2 | C18H30O2 | (9Z,12Z,15Z)- |
| | | | octadeca-9,12,15- |
| | | | trienoic acid |
| MolPort-003-939-239 | −7.7 | C18H34O3 | (9Z,12R)-12- |
| | | | hydroxyoctadec- |
| | | | 9-enoic acid |
| MolPort-001-788-274 | −7.7 | C18H34O2 | (9Z)-octadec-9- |
| | | | enoic acid |
| MolPort-001-788-459 | −7.6 | C18H32O2 | (9Z,12Z)-octadeca- |
| | | | 9,12-dienoic acid |
| MolPort-009-758-577 | −7.1 | C27H45NO6 | [3-(2,2- |
| | | | dimethyloxan-4-yl)-6- |
| | | | methylheptyl]({[4- |
| | | | (propan-2-yloxy) |
| | | | phenyl]]methyl}) amine; |
| | | | oxalic acid |

### Example 4: Experiment on Candidate Compounds in Cells

### Example 4-1: Test of Efficacy of Single Candidate Drugs in Vero Cells

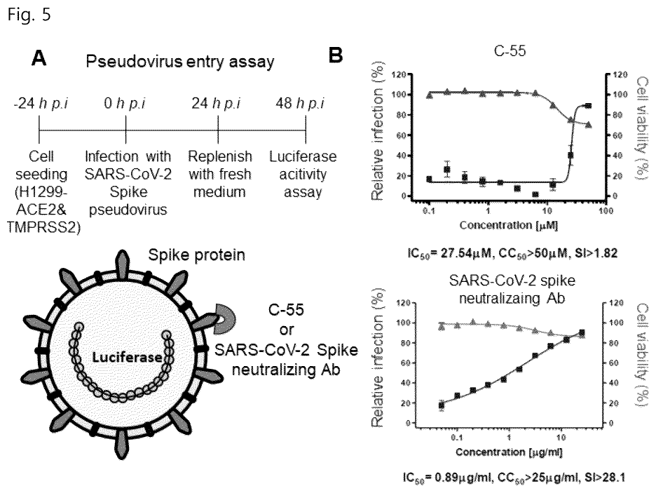

SARS-CoV-2 (βCoV/KOR/KCDC03/2020) and each of a total of 26 drugs containing compounds that bind specifically to the spike proteins selected in Example 3 were injected into Vero cells (monkey kidney cells, purchased from American Type Culture Collection (ATCC)) and then the degree of infection with the virus and the number of cells were quantitatively measured through image analysis using immunofluorescence (Table 3). In both Vero cells (Example 4-1) and Calu-3 cells (Example 4-2), primary antibodies (Sino biological, cat no: 40143-T62), secondary antibodies (Invitrogen, cat no: A-11034), and nuclei staining antibodies (Invitrogen, cat no: H3570) were used for immunoprecipitation. Three types of coronavirus disease 2019 treatments (chloroquine, lopinavir, remdesivir), which are known to have antivirus activity against SARS-CoV-2 in vitro were used as reference drugs (FIG. 5). Vero cells were obtained from the American Type Culture Collection (ATCC CCL-81). SARS-CoV-2 (RBCoV/KOR/KCDC03/2020) was obtained from the Korea Centers for Disease Control and Prevention (KCDC).

TABLE 3

| | Docking | | IC₅₀ | CC₅₀ | |
|---|---|---|---|---|---|
| Compound ID | energy (kcal/mol) | Molecular formula | (μM) | (μM) | Selective Index (CC₅₀/IC₅₀) |
| MolPort-005- | −8.8 | C26H32N2O2 | 8.487 | >100 | 11.782 |
| 980-235(C-55) | | | | | |
| MolPort-027- | −8.3 | C26H28N2O3 | 23.315 | >100 | 4.289 |
| 564-039 | | | | | |
| MolPort-002- | −8.7 | C25H35NO3 | 23.477 | >100 | 4.259 |
| 151-168 | | | | | |
| MolPort-002- | −8.4 | C20H30O5 | 24.608 | >100 | 4.064 |
| 515-990 | | | | | |
| MolPort-003- | −8.3 | C23H38O2 | 29.904 | >100 | 3.344 |
| 939-688 | | | | | |
| MolPort-000- | −9.1 | C23H24O6 | 31.157 | >100 | 3.21 |
| 424-809 | | | | | |
| MolPort-001- | −7.6 | C18H32O2 | 37.640 | >100 | 2.657 |
| 788-459 | | | | | |
| MolPort-003- | −8.8 | C21H24O4 | 43.320 | >100 | 2.308 |
| 709-991 | | | | | |
| MolPort-000- | −9.3 | C27H30N2O6 | 15.611 | 32.435 | 2.078 |
| 829-850 | | | | | |
| MolPort-002- | −9.6 | C23H29NO3S | 28.481 | 57.989 | 2.036 |
| 520-573 | | | | | |
| MolPort-003- | −8.4 | C20H23NO4 | 24.296 | 49.026 | 2.018 |
| 711-757 | | | | | |
| MolPort-027- | −9.3 | C27H33N3O2 | 18.519 | 34.263 | 1.85 |
| 564-475 | | | | | |
| MolPort-009- | −7.1 | C27H45NO6 | 7.941 | 14.523 | 1.829 |
| 758-577 | | | | | |
| MolPort-003- | −8.3 | C20H22BrNO4 | 57.450 | >100 | 1.741 |
| 713-431 | | | | | |
| MolPort-002- | −8.3 | C25H41NO3 | 32.530 | 56.167 | 1.727 |
| 152-403 | | | | | |
| MolPort-003- | −8.2 | C18H30O2 | 58.568 | >100 | 1.707 |
| 937-817 | | | | | |
| MolPort-003- | −8.7 | C20H30O2 | 41.817 | 70.508 | 1.686 |
| 933-180 | | | | | |
| MolPort-003- | −7.7 | C18H34O3 | 60.495 | >100 | 1.653 |
| 939-239 | | | | | |
| MolPort-002- | −9.3 | C28H25N3O | 25.835 | 42.201 | 1.633 |
| 526-760 | | | | | |
| MolPort-001- | −7.7 | C18H34O2 | 67.852 | >100 | 1.474 |
| 788-274 | | | | | |
| MolPort-005- | −9.1 | C25H28FN3O3 | 24.28 | 34.69 | 1.429 |
| 947-745 | | | | | |
| MolPort-005- | −9 | C23H23N3O3 | 71.272 | >100 | 1.403 |
| 974-761 | | | | | |
| MolPort-008- | −9.2 | C22H32O2 | 28.163 | 36.211 | 1.286 |
| 266-638 | | | | | |
| MolPort-002- | −8.5 | C25H25NO6 | 28.730 | 33.365 | 1.161 |
| 526-563 | | | | | |
| MolPort-003- | −9.2 | C22H32O2 | 58.630 | 62.388 | 1.064 |
| 932-890 | | | | | |
| MolPort-003- | −8.2 | C21H26O2S | 94.400 | >100 | 1.059 |
| 713-147 | | | | | |

### Example 4-2: Validation of Single Candidate Drug Performance in Calu-3 Cells

SARS-CoV-2 (βCoV/KOR/KCDC03/2020) and a total of five drugs that exhibited strong antiviral activity in Example 4-1 were injected into Calu-3 cells (human lung cells) and then the degree of virus infection and the number of cells were quantitatively measured through image analysis using immunofluorescence (Table 4).

TABLE 4

| | Docking | | | | Selective |
|---|---|---|---|---|---|
| | energy | Molecular | IC₅₀ | CC₅₀ | Index |
| Compound ID | (kcal/mol) | formula | (μM) | (μM) | (CC₅₀/IC₅₀) |
| MolPort-005- | −8.8 | C26H32N2O2 | 15.566 | >100 | 6.424 |
| 980-235(C-55) | | | | | |
| MolPort-002- | −8.7 | C25H35NO3 | 60.715 | >100 | 1.647 |
| 151-168 | | | | | |
| MolPort-002- | −8.4 | C20H30O5 | 52.182 | >100 | 1.916 |
| 515-990 | | | | | |
| MolPort-003- | −8.3 | C23H38O2 | 83.773 | >100 | 1.194 |
| 939-688 | | | | | |
| MolPort-000- | −9.1 | C23H24O6 | 50.651 | >100 | 1.974 |
| 424-809 | | | | | |

### Example 5: Validation Test of Candidate Substance Drug Mechanism of Action (Inhibition of Virus Entry into Cells)

### Example 5-1: Validation Test of Virus Entry into Cells Based on Pseudovirus

To determine whether or not the MolPort-005-980-235 (C-55) compound derived from Example 4-2 inhibits the entry of SARS-CoV-2 into cells, a pseudovirus-based virus entry verification experiment was performed. The cells used herein were H1299-ACE2-TMPRSS2. The cells were infected with SARS-CoV-2 spike pseudovirus particles containing luciferase and then cultured for 48 hours. Then, luciferase activity was measured in the infected cells to quantitatively determine whether or not the virus entered the cells. SARS-CoV-2 spike neutralizing antibody (Sino biological, #40592-MM57) was used as a reference drug. MolPort-005-980-235 (C-55) was found to have IC50 of 27.54 μM and CC50>50 μM. It was found that, upon treatment with MolPort-005-980-235 (C-55) at a concentration of 50 μM, the intracellular entry of the SARS-CoV-2 virus was inhibited by about 90%.

### Example 5-2: Test to Determine Mechanism of Inhibition of Virus Intracellular Entry Based on Time-of-Addition Assay

To determine whether or not the MolPort-005-980-235 (C-55) compound derived from Example 4-2 inhibits the intracellular entry of SARS-CoV-2, a time-of-addition assay was performed. Vero cells were used as the cell line and infected with the SARS-CoV-2 virus (B CoV/Korea/KCDC03/2020, NCCP43326 Vero_p2_Vero E6_p1) at a multiplicity of infection (MOI) of 3. The cells were treated with 50 μM MolPort-005-980-235 (C-55) at 1-hour intervals before and after virus infection, and the virus infection pattern was observed (observed from −1 to 6 hours). It was found that, when the cells were treated with the compound 1 hour before virus infection or simultaneously with the virus, virus infection did not occur, and virus infection could not be prevented when treated with the drug 1 hour after virus infection.

### Example 6: Validation Test of Efficacy of Candidate Substance in Mice

The preventive and therapeutic effects for SARS-CoV-2 of MolPort-005-980-235 (C-55) derived from Examples 4 and 5 were determined in a mouse animal model. The SARS-CoV-2 virus used herein was βCoV/Korea/KCDC03/2020, NCCP43326 (Vero P3). The mice used herein were 7-week-old males (hACE2 transgenic mice, JAX #034860). The drug was administered once a day for a total of three times, starting 4 hours before virus infection. On the third day, the lungs were autopsied to measure the virus titer (TCID50) and viral RNA.

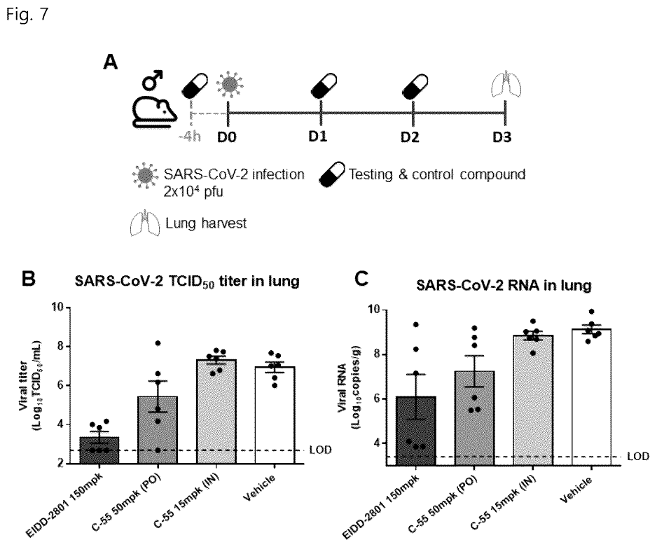

The viral RNA in the lungs in the group administered MolPort-005-980-235 (C-55) orally at 50 mg/kg decreased at a statistically significant level, and the viral titer also tended to decrease (FIG. 7).

### Example 7: Validation Test of Efficacy Against SARS-CoV-2 Variants

The antiviral activity of MolPort-005-980-235 (C-55) derived from Examples 4 and 5 against various SARS-CoV-2 variants, including Omicron, was determined. Vero cells were infected with various SARS-CoV-2 variants and the antiviral activity was evaluated depending on the drug concentration. A total of six mutant viruses were identified and the information is as follows. Ancestral SARS-CoV-2 (lineage A), Alpha (B.1.1.7_UK), Beta (B.1.351_South Africa), Gamma (P.1_Brazil), Delta (B.1.617.2_India), Omicron (B.1.1.529)

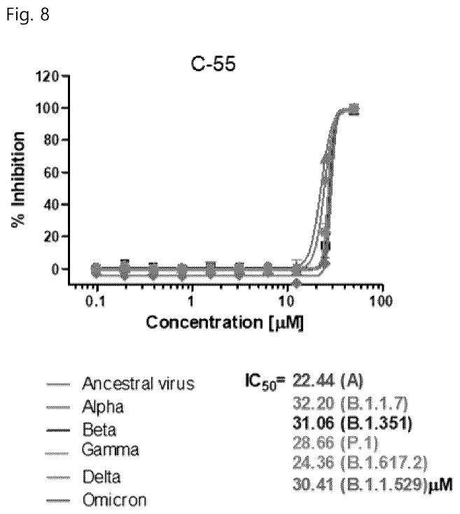

MolPort-005-980-235 (C-55) had antiviral activity against all verified SARS-CoV-2 variants, including Omicron, in a dose-dependent manner (FIG. 8).

### Example 8: Efficacy Test Against Influenza Virus

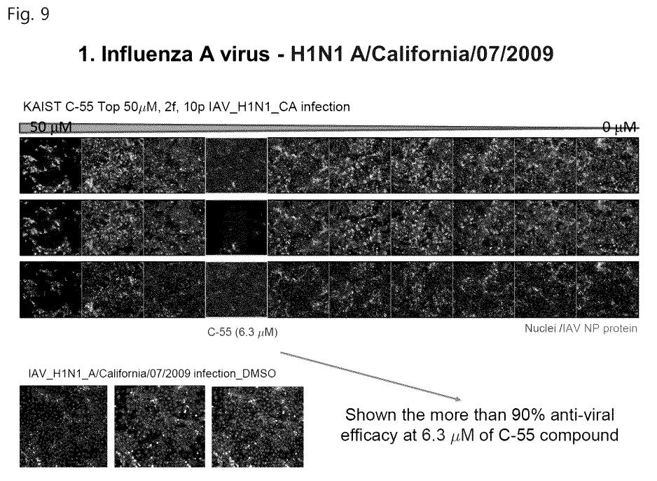

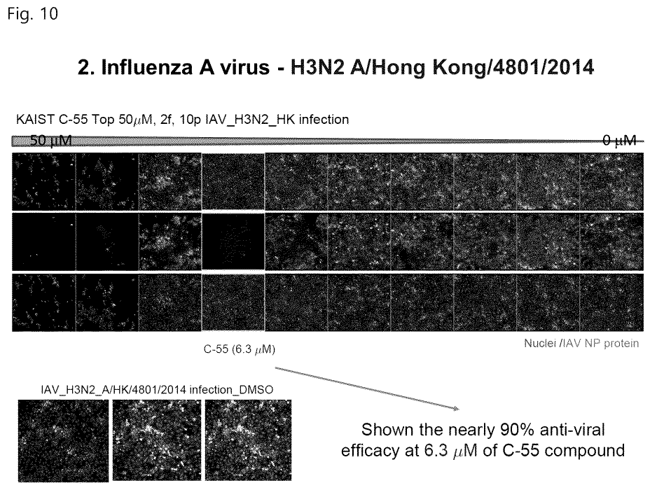

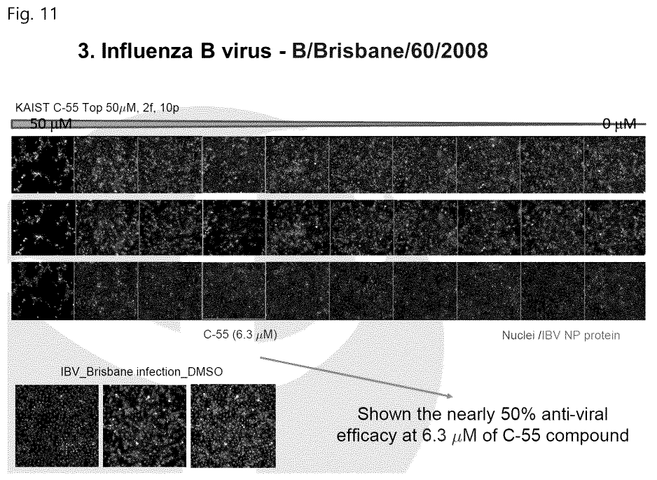

The antiviral activity of MolPort-005-980-235 (C-55) derived from Examples 4 and 5 against influenza virus was determined. Madin-Darby Canine Kidney Cells (MDCK cells), which are canine kidney cells, were used for influenza cell infection. The viruses used herein were two types of influenza A (H1N1 A/California/07/2009, H3N2 A/Hong Kong/4801/2014) and one type of influenza B (B/Brisbane/60/2008). MolPort-005-980-235 (C-55) had antiviral activity against all tested influenza viruses, and in particular, MolPort-005-980-235 (C-55) had about 90% antiviral activity against influenza virus type A, H1N1 A/California/07/2009 and H3N2 A/Hong Kong/4801/2014 when treated at a concentration of 6.3 μM. MolPort-005-980-235 (C-55) had about 50% antiviral activity at a concentration of 6.3 μM against influenza virus type B, B/Brisbane/60/2008 (FIGS. 9 to 11).

### INDUSTRIAL APPLICABILITY

It has already been empirically proven that the antiviral composition for coronavirus or influenza virus according to the present invention and the pharmaceutical composition for preventing or treating viral infection are based on natural products or natural product derivatives and thus have less toxicity than conventional synthetic drugs, and are safe and efficacious upon consumption as food, thus being more safely and effectively useful for preventing or treating virus and viral infections.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method of preventing or treating coronavirus or influenza virus comprising administering a compound selected from the group consisting of the following compounds, a derivative thereof or a salt thereof:
a) [3-(2,2-dimethyloxan-4-yl)-6-methylheptyl]({[4-(propan-2-yloxy)phenyl]methyl})amine and oxalic acid;
b) 10,10-dimethyl-1a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
g) N-[(4-fluorophenyl)methyl]-2-[(3R,4S)-3-{[5-(phenoxymethyl)-1,2-oxazol-3-yl]methyl}piperidin-4-yl]acetamide;
h) 4-[(heptyloxy)carbonyl]phenyl pyridine-3-carboxylate;
i) 2-octyl-1,3-dioxan-5-yl 4-methoxybenzoate;
j) N,1-bis(2-phenylethyl)-9H-pyrido[3,4-b]indole-3-carboxamide;
k) (4Z,7E,10E,13E,16E,19E)-docosa-4,7,10,13,16,19-hexaenoic acid;
l) [(1R,9aR)-octahydro-1H-quinolizin-1-yl]methyl 5-[(benzylsulfanyl)methyl]furan-2-carboxylate;
m) 4-oxo-8-propyl-1H,2H,3H,4H-cyclopenta[c]chromen-7-yl 2-{[(benzyloxy)carbonyl]amino}acetate;
n) Tricosa-10,12-dienoic acid;
o) Ethyl 2-tert-butyl-5-(4-methoxybenzoyloxy)-1-benzofuran-3-carboxylate;
p) 3-(3,5-di-tert-butyl-4-hydroxyphenyl)propyl 2-(cyclohexylamino)acetate;
q) (5Z,8Z,11Z,14Z,17Z)-icosa-5,8,11,14,17-pentaenoic acid;
r) 4-(4-heptylbenzoyloxy)benzoic acid;
s) 4-[(heptyloxy)carbonyl]phenyl 5-bromopyridine-3-carboxylate;
t) (9Z,12Z,15Z)-octadeca-9,12,15-trienoic acid;
u) (4Z,7Z,10Z,13Z,16Z,19Z)-docosa-4,7,10,13,16,19-hexaenoic acid;
v) (9Z,12R)-12-hydroxyoctadec-9-enoic acid;
w) (9Z)-octadec-9-enoic acid;
x) 4-[(1-benzofuran-2-yl)formamido]-N-[2-(1H-indol-3-yl)ethyl]butanamide; and
y) 4-(methylsulfanyl)phenyl 4-heptylbenzoate.

2. The method according to claim 1, wherein the compound is selected from the group consisting of the following compounds:
b) 10,10-dimethyl-10a-[(1E)-2-[3-(pentyloxy)phenyl]ethenyl]-3H,4H,10H,10aH-pyrimido[1,2-a]indol-2-ol;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
i) 2-octyl-1,3-dioxan-5-yl 4-methoxybenzoate;
n) Tricosa-10,12-dienoic acid;
o) Ethyl 2-tert-butyl-5-(4-methoxybenzoyloxy)-1-benzofuran-3-carboxylate; and
r) 4-(4-heptylbenzoyloxy)benzoic acid.

3. The method according to claim 1, wherein the compound is
b) 10,10-dimethyl-10a-[(1E)-2-[3-(pentyloxy)phenyl]ethenyl]-3H,4H,10H,10aH-pyrimido[1,2-a]indol-2-ol.

4. The method according to claim 1, wherein the coronavirus is SARS-CoV-2.

5. The method according to claim 1, wherein the influenza virus is an influenza A virus or an influenza B virus.

6. A pharmaceutical composition for preventing or treating coronavirus or influenza virus comprising a compound selected from the group consisting of the following compounds, a derivative thereof or a salt thereof:
a) [3-(2,2-dimethyloxan-4-yl)-6-methylheptyl]({[4-(propan-2-yloxy)phenyl]methyl})amine and oxalic acid;
b) 10,10-dimethyl-10a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
g) N-[(4-fluorophenyl)methyl]-2-[(3R,4S)-3-{[5-(phenoxymethyl)-1,2-oxazol-3-yl]methyl}piperidin-4-yl]acetamide;
h) 4-[(heptyloxy)carbonyl]phenyl pyridine-3-carboxylate;
i) 2-octyl-1,3-dioxan-5-yl 4-methoxybenzoate;
j) N,1-bis(2-phenylethyl)-9H-pyrido[3,4-b]indole-3-carboxamide;
k) (4Z,7E,10E,13E,16E,19E)-docosa-4,7,10,13,16,19-hexaenoic acid;
l) [(1R,9aR)-octahydro-1H-quinolizin-1-yl]methyl 5-[(benzylsulfanyl)methyl]furan-2-carboxylate;
m) 4-oxo-8-propyl-1H,2H,3H,4H-cyclopenta[c]chromen-7-yl 2-{[(benzyloxy)carbonyl]amino}acetate;
n) Tricosa-10,12-dienoic acid;
o) Ethyl 2-tert-butyl-5-(4-methoxybenzoyloxy)-1-benzofuran-3-carboxylate;
p) 3-(3,5-di-tert-butyl-4-hydroxyphenyl)propyl 2-(cyclohexylamino)acetate;
q) (5Z,8Z,11Z,14Z,17Z)-icosa-5,8,11,14,17-pentaenoic acid;
r) 4-(4-heptylbenzoyloxy)benzoic acid;
s) 4-[(heptyloxy)carbonyl]phenyl 5-bromopyridine-3-carboxylate;
t) (9Z,12Z,15Z)-octadeca-9,12,15-trienoic acid;
u) (4Z,7Z,10Z,13Z,16Z,19Z)-docosa-4,7,10,13,16,19-hexaenoic acid;
v) (9Z,12R)-12-hydroxyoctadec-9-enoic acid;
w) (9Z)-octadec-9-enoic acid;
x) 4-[(1-benzofuran-2-yl)formamido]-N-[2-(1H-indol-3-yl)ethyl]butanamide; and
y) 4-(methylsulfanyl)phenyl 4-heptylbenzoate.

7. The pharmaceutical composition according to claim 6, wherein the pharmaceutical composition comprises a compound selected from the group consisting of the following compounds:
b) 10,10-dimethyl-10a-[(1E)-2-[3-(pentyloxy)phenyl]ethenyl]-3H,4H,10H,10aH-pyrimido[1,2-a]indol-2-ol;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
i) 2-octyl-1,3-dioxan-5-yl 4-methoxybenzoate;
n) Tricosa-10,12-dienoic acid;
o) Ethyl 2-tert-butyl-5-(4-methoxybenzoyloxy)-1-benzofuran-3-carboxylate; and
r) 4-(4-heptylbenzoyloxy)benzoic acid.

8. The pharmaceutical composition according to claim 6, wherein the pharmaceutical composition comprises:
b) 10,10-dimethyl-10a-[(1E)-2-[3-(pentyloxy)phenyl]ethenyl]-3H,4H,10H,10aH-pyrimido[1,2-a]indol-2-ol.

9. The pharmaceutical composition according to claim 6, wherein the coronavirus infection is COVID-19.

10. The pharmaceutical composition according to claim 6, wherein the influenza is infected with an influenza A or B virus.

11. (canceled)

12. An anti-viral composition against coronavirus or influenza virus comprising a compound selected from the group consisting of the following compounds, a derivative thereof or a salt thereof:
a) [3-(2,2-dimethyloxan-4-yl)-6-methylheptyl]({[4-(propan-2-yloxy)phenyl]methyl})amine and oxalic acid;
b) 10,10-dimethyl-10a-[(1E)-2-[3-(pentyloxy)phenyl]ethenyl]-3H,4H,10H,10aH-pyrimido[1,2-a]indol-2-ol;
c) 3-{1-[3-(3-phenoxyphenoxy)propyl]piperidin-2-yl}pyridine and oxalic acid;
d) N-{[(1S,9aR)-octahydro-1H-quinolizin-1-yl]methyl}-2-[6-(benzyloxy)-1H-indol-1-yl]acetamide;
e) N-{[(1S,9aR)-octahydro-1H-quinolizin-1-yl]methyl}-3-(2-oxo-2H-chromen-3-yl)benzamide;
f) 3-(3,5-di-tert-butyl-4-hydroxyphenyl)propyl 2-(phenylamino)acetate;
g) N-[(4-fluorophenyl)methyl]-2-[(3R,4S)-3-{[5-(phenoxymethyl)-1,2-oxazol-3-yl]methyl}piperidin-4-yl]acetamide;
h) 4-[(heptyloxy)carbonyl]phenyl pyridine-3-carboxylate;
i) 2-octyl-1,3-dioxan-5-yl 4-methoxybenzoate;
j) N,1-bis(2-phenylethyl)-9H-pyrido[3,4-b]indole-3-carboxamide;
k) (4Z,7E,10E,13E,16E,19E)-docosa-4,7,10,13,16,19-hexaenoic acid;
l) [(1R,9aR)-octahydro-1H-quinolizin-1-yl]methyl 5-[(benzylsulfanyl)methyl]furan-2-carboxylate;
m) 4-oxo-8-propyl-1H,2H,3H,4H-cyclopenta[c]chromen-7-yl 2-{[(benzyloxy)carbonyl]amino}acetate;
n) Tricosa-10,12-dienoic acid;
o) Ethyl 2-tert-butyl-5-(4-methoxybenzoyloxy)-1-benzofuran-3-carboxylate;
p) 3-(3,5-di-tert-butyl-4-hydroxyphenyl)propyl 2-(cyclohexylamino)acetate;
q) (5Z,8Z,11Z,14Z,17Z)-icosa-5,8,11,14,17-pentaenoic acid;
r) 4-(4-heptylbenzoyloxy)benzoic acid;
s) 4-[(heptyloxy)carbonyl]phenyl 5-bromopyridine-3-carboxylate;
t) (9Z,12Z,15Z)-octadeca-9,12,15-trienoic acid;
u) (4Z,7Z,10Z,13Z,16Z,19Z)-docosa-4,7,10,13,16,19-hexaenoic acid;
v) (9Z,12R)-12-hydroxyoctadec-9-enoic acid;
w) (9Z)-octadec-9-enoic acid;
x) 4-[(1-benzofuran-2-yl)formamido]-N-[2-(1H-indol-3-yl)ethyl]butanamide; and
y) 4-(methylsulfanyl)phenyl 4-heptylbenzoate.

13. (canceled)
